(19) United States
(12) Patent Application Publication
(10) Pub. No.: US 2026/0002115 A1
(43) Pub. Date: Jan. 1, 2026

(54) NOVEL BACILLUS VELEZENSIS STRAIN PRODUCING MORANOLINE AND USES THEREOF

(71) Applicant: AMOREPACIFIC CORPORATION, Seoul (KR)

(72) Inventors: Hyuk KIM, Yongin-si (KR); Yujin OH, Yongin-si (KR); Kilsun MYOUNG, Yongin-si (KR); Daejin MIN, Yongin-si (KR); Heungsoo BAEK, Yongin-si (KR); Hyunjung CHOI, Yongin-si (KR); Baek Rock OH, Daejeon (KR); Sun-Yeon HEO, Daejeon (KR); Youngbin PARK, Daejeon (KR); Jung-Hyun JU, Daejeon (KR)

(73) Assignee: AMOREPACIFIC CORPORATION, Seoul (KR)

(21) Appl. No.: 19/235,772

(22) Filed: Jun. 12, 2025

(30) Foreign Application Priority Data
Jun. 28, 2024 (KR) 10-2024-0085344

(51) Int. Cl. C12N 1/20; A61K 8/60; A61K 8/99; A61Q 19/02; C12N 9/90; C12P 17/12

(52) U.S. Cl. CPC C12N 1/20; A61K 8/60; A61K 8/99; A61Q 19/02; C12N 9/90; C12P 17/12

(57) ABSTRACT

One aspect of the present disclosure provides a novel Bacillus velezensis strain, a composition for producing moranoline, including the strain, a composition for skin brightening, including the strain, a medium composition for culturing the strain, and a method for producing moranoline, the method including culturing the strain. The Bacillus velezensis strain according to the present invention has a higher amount of moranoline produced than strains known in the related art, and thus, is highly industrially applicable.

### CROSS REFERENCE TO RELATED APPLICATION

The present application claims priority to Korean Patent Application No. 10-2024-0085344, filed Jun. 28, 2024, the entire contents of which are hereby incorporated by this reference.

### REFERENCE TO AN ELECTRONIC SEQUENCE LISTING

The contents of the electronic sequence listing (P2024-00042_Sequence listing.xml; Size: 13,868 bytes; and Date of Creation: May 28, 2025) is herein incorporated by reference in its entirety.

### BACKGROUND OF THE INVENTION

### Field of the Invention

The present invention relates to a novel Bacillus velezensis strain producing moranoline and a use thereof.

### Description of the Related Art

α-Glucosidase is an essential enzyme in vivo, which promotes the cleavage of α-glycosidic bonds that release α-glucose from carbohydrates in the small intestine, and is involved in the absorption of glucose. Further, since α-glucosidase is also involved in various metabolic pathways such as the synthesis of glycolipids and glycoproteins, the regulation thereof may open the possibility of treating metabolic diseases. α-Glucosidase inhibitors are used to treat type 2 diabetes, and Acarbose isolated from Actinoplanes strain SE50 is a potent α-glucosidase inhibitor, and has been shown to reduce postprandial blood glucose levels and promotes insulin secretion.

Tyrosinase, an enzyme involved in the most important initial rate-determining step in the melanin biosynthesis pathway, is a type 1 membrane glycoprotein, and is produced by the N-linked glycosylation process, which attaches sugars to the NH₂ group on the side chain of asparagine, and the N-linked glycosylation process in the O-linked glycosylation process, which attaches sugars to the OH group on the side chains of serine or threonine. N-linked glycosylation of proteins occurs cotranslationally with protein synthesis in the endoplasmic reticulum (ER). 14 sugars (Glc3Man9GlcNAc2), half of the glycan units, are synthesized in the cytoplasm, outside the ER membrane, and the other half is then synthesized in the ER and transferred to proteins. In the ER, this synthesis process begins with the cleavage of three glucose units from the mannose present at the terminal end of oligosaccharides by α-glucosidases I and II. Therefore, when α-glucosidase is inhibited, the glycosylation of tyrosinase is inhibited, and the structure of tyrosinase is transformed and it migrates to melanosomes in an inactive form, resulting in the suppression of melanogenesis.

Moranoline (1-Deoxynojirimycin) may be chemically synthesized or extracted from mulberry trees (Morus alba barks). However, since these methods have a low production yield and are difficult to secure raw materials, they have a problem of being ineffective in terms of cost.

### SUMMARY OF THE INVENTION

The present inventors have discovered a novel Bacillus velezensis strain that produces a larger amount of moranoline than Bacillus strains previously discovered, and have identified optimal culture conditions for mass-producing moranoline.

Therefore, in one aspect, an object of the present invention is to provide a newly discovered Bacillus velezensis AmoreLumina strain.

In one aspect, an object of the present invention is to provide a composition for producing moranoline, including: a Bacillus velezensis AmoreLumina strain; a lysate thereof, a culture solution thereof; or an extract of the culture solution as an active ingredient.

In one aspect, an object of the present invention is to provide a composition for skin brightening, including: a Bacillus velezensis AmoreLumina strain; a lysate thereof, a culture solution thereof, or an extract of the culture solution as an active ingredient.

In one aspect, an object of the present invention is to provide a medium composition for culturing a Bacillus velezensis strain, including sorbitol as a carbon source and soybean meal as a nitrogen source.

In one aspect, an object of the present invention is to provide a method for producing moranoline, the method including culturing a Bacillus velezensis strain, wherein the step is performed with stirring at a speed of 250 rpm to 450 rpm.

In one aspect, there may be provided a Bacillus velezensis AmoreLumina strain deposited under Accession Number KCTC 15911BP.

In an exemplary embodiment, the strain may have DNA encoding a gyrA gene including a sequence of SEQ ID NO: 10.

In one aspect, there may be provided a composition for producing moranoline, including: a Bacillus velezensis AmoreLumina strain deposited under Accession Number KCTC 15911BP; a lysate thereof, a culture solution thereof, or an extract of the culture solution as an active ingredient.

In an exemplary embodiment, the composition may suppress the production of α-glucosidase.

In one aspect, there may be provided a composition for skin brightening, including: a Bacillus velezensis AmoreLumina strain deposited under Accession Number KCTC 15911BP; a lysate thereof, a culture solution thereof, or an extract of the culture solution as an active ingredient.

In an exemplary embodiment, the active ingredient may be a culture solution extract of the strain, and the extract may be used in a daily dosage of 10 mg/kg to 25,000 mg/kg.

In an exemplary embodiment, the composition may suppress the production of melanin.

In an exemplary embodiment, the composition may be a cosmetic or food composition.

In an exemplary embodiment, the composition may be a pharmaceutical composition for preventing or treating a hyperpigmentation disorder.

In one aspect, there may be provided a medium composition for culturing a Bacillus velezensis strain, including sorbitol as a carbon source and soybean meal as a nitrogen source.

In an exemplary embodiment, the pH of the composition may be 5 to 10.

In an exemplary embodiment, the sorbitol may be present in an amount of more than 40 g/L and less than 120 g/L based on the total volume of the composition.

In an exemplary embodiment, the soybean meal may be present in an amount of more than 20 g/L and less than 50 g/L based on the total volume of the composition.

In an exemplary embodiment, the medium composition may include 60 g/L to 100 g/L sorbitol, 30 g/L to 40 g/L soybean meal, 0.1 g/L to 1.0 g/L KH₂PO₄, and 0.1 g/L to 1.0 g/L (NH₄)₂SO₄.

In an exemplary embodiment, the medium may be for producing moranoline.

In an exemplary embodiment, the strain may be a strain deposited under Accession Number KCTC 15911BP.

In one aspect, there may be provided a method for producing moranoline, the method including culturing a Bacillus velezensis strain, wherein the step is performed with stirring at a speed of 250 rpm to 450 rpm.

In an exemplary embodiment, the method may further include adjusting the pH of a strain culture solution obtained after the culture to 4 or less, and filtering the strain culture solution to purify moranoline.

In an exemplary embodiment, the method may further include adjusting the pH of an eluate from the filtered filtrate to a pH of 5.5 to 7.5 and fractionating the eluate.

In an exemplary embodiment, the culturing step may be performed under the condition of a pH of 6.5 to 8.5 for 40 hours or more.

In an exemplary embodiment, the strain may be a strain deposited under Accession Number KCTC 15911BP.

In one aspect, the strain of the present invention; a lysate thereof, a culture solution thereof; or an extract of the culture solution can mass-produce moranoline.

In one aspect, the present invention contributes to reducing the production costs of moranoline.

In one aspect, the strain of the present invention; a lysate thereof, a culture solution thereof; or an extract of the culture solution has excellent skin brightening effects.

In one aspect, the medium composition of the present invention provides optimal culture conditions for the mass-production of moranoline by the strain of the present invention.

### BRIEF DESCRIPTION OF THE DRAWINGS

FIG. 1 shows the HPLC chromatogram results of Moranoline-FOMC derivatization according to an example.

FIG. 2 shows the PCR results of three moranoline biosynthesis-related genes according to an example.

FIG. 3 shows the qualitative analysis results of the culture solution of a newly isolated 10-18 strain using LC-MSMS according to an example.

FIG. 4A shows the results of analyzing the 16S rRNA sequence and phylogenetic tree of a newly isolated B. velezensis AmoreLumina strain according to an example.

FIG. 4B shows a comparison of a portion of the gyrA gene of a newly isolated B. velezensis AmoreLumina strain according to an example with a portion of the gyrA gene of the corresponding comparative strain, Bacillus velezensis strain NY12-2 (′: gyrA gene of B. velezensis AmoreLumina strain, ‘ ’: gyrA gene of Bacillus velezensis strain NY12-2).

FIG. 5 shows a colony photograph (left) and an optical microscope photograph (right, 400× magnification) of the newly isolated B. velezensis AmoreLumina strain according to an example.

FIG. 6 shows the results of culturing the B. velezensis AmoreLumina strain depending on the initial pH according to an example.

FIG. 7 shows the results of producing moranoline by the B. velezensis AmoreLumina strain depending on the type of carbon source according to an example.

FIG. 8 shows the results of producing moranoline by the B. velezensis AmoreLumina strain depending on the type of nitrogen source according to an example.

FIG. 9 shows the results of producing moranoline after 48 hours of culture of the B. velezensis AmoreLumina strain depending on the carbon source concentration according to an example.

FIG. 10 shows the results of producing moranoline after 72 hours of culture of the B. velezensis AmoreLumina strain depending on the nitrogen source concentration according to an example.

FIG. 11 shows the results of culturing the B. velezensis AmoreLumina strain using the optimal medium according to an example.

FIG. 12 is a culture photograph of the B. velezensis AmoreLumina strain using a 5 L fermenter according to an example.

FIG. 13 shows the results of culturing the B. velezensis AmoreLumina strain depending on the stirring speed according to an example.

FIG. 14 shows the results of analyzing the content of moranoline in the B. velezensis AmoreLumina strain culture solution according to an example.

FIG. 15 shows the results of analyzing the content of moranoline before and after purification of the B. velezensis AmoreLumina strain culture solution extract according to an example.

FIG. 16 shows the results of comparing the AGI activities of the B. velezensis AmoreLumina strain culture solution extract according to an example and moranoline.

FIG. 17 shows the results of comparing the melanin synthesis inhibitory efficacies of the B. velezensis AmoreLumina strain culture solution extract according to an embodiment and moranoline.

FIG. 18 shows the results of evaluating the brightening efficacy of a culture solution extract of the B. velezensis AmoreLumina strain in an artificial skin according to an example.

### DETAILED DESCRIPTION OF THE INVENTION

Hereinafter, the present invention will be described in detail.

In one aspect, the present invention may provide a Bacillus velezensis AmoreLumina strain deposited under Accession Number KCTC 15911BP.

The Bacillus velezensis AmoreLumina strain deposited under Accession Number KCTC 15911BP is a novel strain isolated and identified to produce moranoline from fermented soybean foods, and was assigned the Accession Number KCTC 15911BP on May 20, 2024.

The amount of moranoline produced by the Bacillus velezensis AmoreLumina strain may be remarkably improved compared to known strains with the same attributes.

In an exemplary embodiment, the strain may have DNA encoding a gyrA gene including a sequence of SEQ ID NO:10. The gyrA gene including the sequence of SEQ ID NO:10 may be a part of the gyrA gene of the Bacillus velezensis strain. The sequence of SEQ ID NO:10 may differ from the gyrA gene sequences of the known Bacillus velezensis strains in one or more sequences. The gyrA gene may be a housekeeping gene. Since housekeeping genes are genes that perform basic cell functions and means genes expressed under all circumstances, they may be important within each individual or species.

In another aspect, the present invention may provide a method for producing moranoline, the method including culturing a composition including: a Bacillus velezensis AmoreLumina strain deposited under Accession Number KCTC 15911BP; a lysate thereof, a culture solution thereof, or an extract of the culture solution as an active ingredient. In addition, the present invention may provide a composition for producing moranoline, including: a Bacillus velezensis AmoreLumina strain deposited under Accession Number KCTC 15911BP; a lysate thereof, a culture solution thereof, or an extract of the culture solution as an active ingredient.

The description on the Bacillus velezensis AmoreLumina strain deposited under Accession Number KCTC 15911BP is as described above.

The lysate may refer to a product obtained by crushing the strain itself by chemical or physical force.

The culture solution may refer to a material including all materials included in a medium in which the strain is cultured, for example, a material including metabolites or secretions resulting from the culture of the strain, or a lysate thereof, and the strain itself may also be included in the culture solution.

The extract may refer to a product obtained by extracting the strain itself, a lysate of the strain, a culture solution of the strain, an apoptotic body of the strain, or a mixture thereof using an extraction solvent.

The moranoline (1-deoxynojirimycin) refers to a polyhydroxyl alkaloid in which oxygen in a glucose structure is substituted with nitrogen.

In an exemplary embodiment, the active ingredient may suppress the production of α-glucosidase. The present inventors have confirmed that the composition of the present invention has excellent α-glucosidase inhibitory activity using an AGI activity evaluation method, and thus, the amount of moranoline produced is remarkable.

In still another aspect, the present invention may provide a method for brightening the skin, the method including administering, to a subject in need of skin brightening, a composition including: a Bacillus velezensis AmoreLumina strain deposited under Accession Number KCTC 15911BP; a lysate thereof, a culture solution thereof, or an extract of the culture solution as an active ingredient. Furthermore, the present invention may provide a composition for skin brightening, including: a Bacillus velezensis AmoreLumina strain deposited under Accession Number KCTC 15911BP; a lysate thereof, a culture solution thereof, or an extract of the culture solution as an active ingredient.

The description on the Bacillus velezensis AmoreLumina strain deposited under Accession Number KCTC 15911BP, lysate, culture solution and extract is as described above.

A composition including the novel strain of the present invention, a lysate thereof, a culture solution thereof, or an extract of the culture solution as an active ingredient may suppress the expression of factors that have a negative effect on skin brightening. Specifically, the present inventors have confirmed that the composition of the present invention has an excellent effect of suppressing melanin synthesis, and has a remarkable effect on skin brightening by suppressing pigmentation in the epidermis layer of the skin.

In an exemplary embodiment, the active ingredient is a culture solution extract of the strain, and the extract may be used in a daily dosage of 10 mg/kg to 25,000 mg/kg. Further, the extract may be used in a daily dosage of 10 mg/kg or more, 50 mg/kg or more, 100 mg/kg or more, 200 mg/kg or more, 300 mg/kg or more, 400 mg/kg or more, 500 mg/kg or more, 1,000 mg/kg or more, 1,250 mg/kg or more, 1,500 mg/kg or more, 1,750 mg/kg or more, 2,000 mg/kg or more, 2,250 mg/kg or more, 2,500 mg/kg or more, 2,750 mg/kg or more, 3,000 mg/kg or more, 3,250 mg/kg or more, 3500 mg/kg or more, 3,750 mg/kg or more, 4,000 mg/kg or more, 4,250 mg/kg or more, 4,500 mg/kg or more, or 4,750 mg/kg or more, and the daily dosage is not limited thereto. In addition, the extract may be used in a daily dosage of 25,000 mg/kg or less, 24,000 mg/kg or less, 23,000 mg/kg or less, 22,000 mg/kg or less, 21,000 mg/kg or less, 20,000 mg/kg or less, 19,000 mg/kg or less, 18,000 mg/kg or less, 17,000 mg/kg or less, 16,000 mg/kg or less, 15,000 mg/kg or less, 14,000 mg/kg or less, 13,000 mg/kg or less, 12,000 mg/kg or less, 11,000 mg/kg or less, 10,000 mg/kg or less, 9,500 mg/kg or less, 9,000 mg/kg or less, 8,500 mg/kg or less, 8,000 mg/kg or less, 7,500 mg/kg or less, 7,000 mg/kg or less, 6,500 mg/kg or less, 6,000 mg/kg or less, 5,750 mg/kg or less, 5,500 mg/kg or less, or 5,250 mg/kg or less, but the daily dosage is not limited thereto.

In an exemplary embodiment, the brightening may suppress the production of melanin. The present inventors have confirmed that melanin synthesis in human melanocytes may be suppressed with high efficiency.

In an exemplary embodiment, the composition may be a cosmetic or food composition.

The appearance of the cosmetic composition may contain a cosmetically or dermatologically acceptable medium or base. It is any formulation suitable for topical application, and may be provided in the form of, for example, a solution, a gel, a solid, a kneaded anhydrous product, an emulsion obtained by dispersing an oil phase in the form of an aqueous phase, a suspension, a microemulsion, a microcapsule, microgranules or an ionic (liposome) and a non-ionic vesicular dispersing agent, or in the form of a cream, a skin toner, a lotion, a powder, an ointment, a spray, or a conceal stick. These compositions may be prepared by a typical method in the field. The composition according to the present specification may also be prepared in the form of a foam or an aerosol composition that further contains a compressed propellant.

The cosmetic composition is not particularly limited in its formulation, and may be formulated into a cosmetic such as, for example, a softening lotion, an astringent lotion, a nourishing lotion, a nourishing cream, a massage cream, an essence, an eye cream, an eye essence, a cleansing cream, a cleansing foam, a cleansing water, a pack, a powder, a body lotion, a body cream, a body oil, or a body essence.

When the formulation of the cosmetic composition is a paste, a cream or a gel, an animal oil, a vegetable oil, a wax, paraffin, starch, traganth, a cellulose derivative, a polyethylene glycol, silicone, bentonite, silica, talc, zinc oxide, or the like may be used as a carrier ingredient.

When the formulation of the cosmetic composition is a powder or a spray, lactose, talc, silica, aluminum hydroxide, calcium silicate, or a polyamide powder may be used as the carrier ingredient, and in particular, when the formulation of the present invention is a spray, the formulation may additionally include a propellant such as a chlorofluorohydrocarbon, propane/butane or dimethyl ether.

When the formulation of the cosmetic composition is a solution or an emulsion, a solvent, a solubilizer or an emulsifier may be used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the cosmetic composition is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, traganth, or the like may be used as a carrier ingredient.

The cosmetic composition according to an exemplary embodiment may further include functional additives and ingredients included in general cosmetic compositions in addition to the extract including the active ingredient. The functional additive may include one or more ingredients selected from the group consisting of a water-soluble vitamin, an oil-soluble vitamin, a polymeric peptide, a polymeric polysaccharide, a sphingolipid, and a seaweed extract.

The cosmetic composition may be blended with ingredients included in general cosmetic compositions, if necessary, along with the functional additives. Examples of other blended ingredients which may be contained include oil and fat ingredients, a moisturizer, an emollient, a surfactant, organic and inorganic pigments, an organic powder, an ultraviolet absorbent, a preservative, a bactericide, an antioxidant, a plant extract, a pH adjuster, an alcohol, a colorant, a fragrance, a circulation accelerator, a cooling agent, an antiperspirant, purified water, and the like.

A formulation of the food composition is not particularly limited, but the food composition may be formulated into, for example, a tablet, a granule, a pill, a powder, a liquid such as a drink, a caramel, a gel, a bar, a tea bag, or the like. For the food composition of each formulation, ingredients typically used in the art may be appropriately selected and compounded by a person with ordinary skill in the art according to the formulation or purpose of use, in addition to the active ingredient, and when the food composition is simultaneously applied with other raw materials, a synergistic effect may occur.

The food composition may include various nutrients, vitamins, minerals (electrolytes), flavoring agents such as synthetic flavoring agents and natural flavoring agents, colorants and fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in a carbonated beverage, or the like. Furthermore, the food compositions according to an embodiment may include flesh of fruit for the preparation of natural fruit juices and fruit juice drinks and vegetable drinks. These ingredients may be used either alone or in combination. The proportion of these additives is not significantly important, but is generally included within a range of 0 to 50 parts by weight per 100 parts by weight of the composition according to an embodiment.

When composition is used as a food additive, the composition may be added as it is or used with another food or other food ingredients, and may be appropriately used according to a typical method. An amount of active ingredient mixed may be determined by a typical method in the art according to the purpose of use.

In an exemplary embodiment, the brightening may be the prevention or treatment of a hyperpigmentation disorder. The hyperpigmentation disorder may be a melanin hyperpigmentation disorder. The melanin hyperpigmentation disorder may be lentigo, melasma, freckles, age spots, or solar lentigines, but is not limited thereto.

The pharmaceutical composition may further contain a pharmaceutical adjuvant such as a preservative, stabilizer, hydrating agent, or an emulsion-promoting agent, a salt and/or buffer for adjusting osmotic pressure, and other therapeutically useful materials, and may be formulated into various formulations for oral or parenteral administration by typical methods.

Examples of the formulation for oral administration include a tablet, a pill, a hard/soft capsule, a solution, a suspension, an emulsion, a syrup, a powder, a dust, a fine granule, a granule, a pellet, and the like, and these formulations contain a surfactant, a diluent (for example: lactose, dextrose, sucrose, mannitol, sorbitol, cellulose and glycine) and a lubricant (for example: silica, talc, stearic acid, and a magnesium or calcium salt thereof, and polyethylene glycol) in addition to the active ingredient. The tablet may also contain a binder such as magnesium aluminum silicate, starch paste, gelatin, tragacanth, methyl cellulose, sodium carboxymethyl cellulose, and polyvinylpyrrolidine, and may contain a pharmaceutical additive such as a disintegrant such as starch, agar, alginic acid, or a sodium salt thereof, an absorbent, a colorant, a flavor, a sweetener, and the like in some cases. The tablet may be prepared by a typical mixing, granulation or coating method. Further, the formulation for parenteral administration may be a rectal, topical, subcutaneous, or transdermal administration type formulation, and may be a formulation such as, for example, an injection, a drop, an ointment, a lotion, a gel, a cream, a spray, a suspension, an emulsion, a suppository, or a patch, but is not limited thereto. The pharmaceutical composition according to an embodiment of the present specification may be administered topically, for example, to the skin.

Determination of the administration dosage of the active ingredient is within the level of the person skilled in the art, a daily administration dosage of a drug may vary depending on various factors such as the progression of symptoms, the time of onset, age, health status, and complications of a subject to be administered, but based on an adult, the drug may generally be administered in divided doses 1 to 3 times a day, and the administration dosage does not limit the scope of the present specification by any means.

In yet another aspect, there may be provided a medium composition for culturing a Bacillus velezensis strain, including sorbitol as a carbon source and soybean meal as a nitrogen source.

The present inventors have confirmed that when sorbitol was used as a carbon source in the medium composition for culturing a Bacillus velezensis strain, the strain herein was able to produce the highest level of moranoline compared to a medium using glucose, fructose, sucrose or galactose. In addition, the present inventors have confirmed that when soybean mean was used as a nitrogen source in the medium composition, the strain herein was able to produce the highest level of moranoline compared to a medium using yeast extract, soy peptone or peptone.

In an exemplary embodiment, the pH of the composition may be 5 to 10. The pH of the composition may be 5 or more, 5.2 or more, 5.4 or more, 5.6 or more, 5.8 or more, 6 or more, 6.2 or more, 6.4 or more, 6.6 or more, 6.8 or more, 7.0 or more, 7.1 or more, 7.2 or more, 7.3 or more, 7.4 or more, or 7.45 or more, but is not limited thereto. Furthermore, the pH of the composition may be 10 or less, 9.8 or less, 9.6 or less, 9.4 or less, 9.2 or less, 9 or less, 8.9 or less, 8.7 or less, 8.6 or less, 8.5 or less, 8.4 or less, 8.3 or less, 8.2 or less, 8.1 or less, 8.0 or less, 7.9 or less, 7.8 or less, 7.7 or less, 7.65 or less, 7.6 or less, or 7.55 or less, but is not limited thereto.

In an exemplary embodiment, the sorbitol may be present in an amount of more than 40 g/L and less than 120 g/L based on the total volume of the composition. The sorbitol may be present in an amount of more than 40 g/L, more than 42 g/L, more than 44 g/L, more than 46 g/L, more than 48 g/L, more than 50 g/L, more than 52 g/L, more than 54 g/L, more than 56 g/L, or more than 58 g/L based on the total volume of the composition, but the amount is not limited thereto. Further, the sorbitol may be present in an amount of less than 120 g/L, less than 118 g/L, less than 116 g/L, less than 114 g/L, less than 112 g/L, less than 110 g/L, less than 108 g/L, less than 106 g/L, less than 104 g/L, or less than 102 g/L based on the total volume of the composition, but the amount is not limited thereto.

In an exemplary embodiment, the soybean meal may be present in an amount of more than 20 g/L and less than 50 g/L based on the total volume of the composition. The soybean meal may be present in an amount of more than 20 g/L, more than 21 g/L, more than 22 g/L, more than 23 g/L, more than 24 g/L, more than 25 g/L, more than 26 g/L, more than 27 g/L, more than 27.5 g/L, more than 28 g/L, more than 28.5 g/L, more than 29 g/L, or more than 29.5 g/L based on the total volume of the composition, but the amount is not limited thereto. In addition, the soybean meal may be present in an amount of less than 50 g/L, less than 49 g/L, less than 48 g/L, less than 47 g/L, less than 46 g/L, less than 45 g/L, less than 44 g/L, less than 43 g/L, less than 42.5 g/L, less than 42 g/L, less than 41.5 g/L, less than 41 g/L, or less than 40.5 g/L based on the total volume of the composition, but the amount is not limited thereto.

In an exemplary embodiment, the medium composition may include 60 g/L to 100 g/L sorbitol, 30 g/L to 40 g/L soybean meal, 0.1 g/L to 1.0 g/L KH₂PO₄, and 0.1 g/L to 1.0 g/L (NH₄)₂SO₄. The present inventors have confirmed through the experimental examples of the present invention that when 60 g/L to 100 g/L sorbitol and 30 g/L to 40 g/L soybean mean were included in the medium composition, the amount of moranoline produced is particularly excellent. Furthermore, the KH₂PO₄ or (NH₄)₂SO₄ may be included in an amount of 0.1 g/L or more, 0.15 g/L or more, 0.2 g/L or more, 0.25 g/L or more, 0.3 g/L or more, 0.35 g/L or more, 0.4 g/L or more, or 0.45 g/L or more, but the amount is not limited thereto. Further, the KH₂PO₄ or (NH₄)₂SO₄ may be included in an amount of 1.0 g/L or less, 0.95 g/L or less, 0.9 g/L or less, 0.85 g/L or less, 0.8 g/L or less, 0.75 g/L or less, 0.7 g/L or less, 0.65 g/L or less, 0.6 g/L or less, or 0.55 g/L or less, but the amount is not limited thereto.

In an exemplary embodiment, the medium may be for producing moranoline.

In an exemplary embodiment, the strain may be a strain deposited under Accession Number KCTC 15911BP. The medium composition may maximize the production efficiency of moranoline by the Bacillus velezensis AmoreLumina strain of the present invention. The present inventors have determined the levels of each factor in the culture medium composition that is the most effective for moranoline production because the amount of moranoline produced by the strain herein varies depending on the pH conditions of the medium composition and the types and concentrations of carbon and nitrogen sources.

In still yet another aspect, the present invention may provide a method for producing moranoline, the method including culturing a Bacillus velezensis strain, wherein the step is performed with stirring at a speed of 250 rpm to 450 rpm.

The stirring speed in the culturing step may be 250 rpm to 450 rpm. The present inventors have confirmed the most effective stirring speed for moranoline production because the amount of moranoline produced by the strain varies depending on the stirring speed conditions in the culturing step. For example, the stirring speed in the culturing step may be 250 rpm or more, 255 rpm or more, 260 rpm or more, 265 rpm or more, 270 rpm or more, 275 rpm or more, 280 rpm or more, 285 rpm or more, 290 rpm or more, or 295 rpm or more, but is not limited thereto. In addition, the stirring speed in the culturing step may be 450 rpm or less, 445 rpm or less, 440 rpm or less, 435 rpm or less, 430 rpm or less, 425 rpm or less, 420 rpm or less, 415 rpm or less, 410 rpm or less, or 405 rpm or less, but is not limited thereto.

In an exemplary embodiment, the method may further include adjusting the pH of a strain culture solution obtained after the culture to 4 or less, and filtering the strain culture solution to purify moranoline. In the purifying step, the pH of the strain culture solution may be adjusted to 4 or less, 3.9 or less, or less, 3.8 or less, 3.7 or less, 3.6 or less, 3.5 or less, 3.4 or less, 3.3 or less, 3.2 or less, or 3.1 or less, but is not limited thereto. Furthermore, the filtering method may further include, without limitation, a method of filtering moranoline from a strain culture solution known in the art.

In an exemplary embodiment, the method may further include adjusting the pH of an eluate from the filtered filtrate to a pH of 5.5 to 7.5 and fractionating the eluate. In the fractionating step, the pH of the eluate may be adjusted to 5.5 or more, 5.55 or more, 5.6 or more, 5.65 or more, 5.7 or more, 5.75 or more, 5.8 or more, 5.85 or more, 5.9 or more, or 5.95 or more, but is not limited thereto. Further, the pH of the eluate may be adjusted to 7.5 or less, 7.45 or less, 7.4 or less, 7.35 or less, 7.3 or less, 7.25 or less, 7.2 or less, 7.15 or less, 7.1 or less, or 7.05 or less, but is not limited thereto.

The present inventors have confirmed that the strain culture solution before purification was dark brown in color and had a distinctive unpleasant odor, but after the purification method of the present invention was performed, the culture solution became transparent brown in color and the distinctive odor was greatly reduced.

In an exemplary embodiment, the culturing step may be performed under the condition of a pH of 6.5 to 8.5 for 40 hours or more. The pH condition in the culturing step may be 6.5 or more, 6.6 or more, 6.7 or more, 6.8 or more, 6.9 or more, 7 or more, 7.1 or more, 7.2 or more, 7.3 or more, 7.4 or more, but is not limited thereto. In addition, the pH in the culturing step may be 8.5 or less, 8.4 or less, 8.3 or less, 8.2 or less, 8.1 or less, 8 or less, 7.9 or less, 7.8 or less, 7.7 or less, or 7.6 or less, but is not limited thereto. Furthermore, the culturing step may be performed for, but is not limited to, 40 hours or more, 45 hours or more, 50 hours or more, 51 hours or more, 52 hours or more, 53 hours or more, 54 hours or more, 55 hours or more, 56 hours or more, 57 hours or more, 58 hours or more, 59 hours or more, 60 hours or more, 61 hours or more, 62 hours or more. Further, the culturing step may be performed for, but is not limited to, 100 hours or less, 97.5 hours or less, 95 hours or less, 92.5 hours or less, 90 hours or less, 89 hours or less, 88 hours or less, 87 hours or less, 86 hours or less, 85 hours or less, 84 hours or less, 83 hours or less, or 82 hours or less.

In an exemplary embodiment, the strain may be a strain deposited under Accession Number KCTC 15911BP.

### EXAMPLES

### 1. Isolation and Selection of Novel Moranoline-Producing Microorganism

### 1) Isolation of Novel Moranoline-Producing Microorganism

20 samples of each of doenjang and cheonggukjang, Korean fermented soybean foods, were secured from various sources. The 40 secured samples were serially diluted, smeared on an LB solid plate medium (10 g/L tryptone, 5 g/L yeast extract, 10 g/L sodium chloride, and 15 g/L agar), and incubated in a 37° C. incubator for 24 hours. A total of 2,000 or more single colonies were secured by pure isolation, and the secured single colonies were cultured in liquid culture using a GY30 medium (30 g/L glucose, 10 g/L yeast extract, 0.5 g/L KH₂PO₄, and 0.5 g/L (NH₄)₂SO₄). The single colonies were cultured in liquid culture for 72 hours under the conditions of a culture temperature of 37° C. and a stirring speed of 200 rpm using a 50 mL conical tube including 10 mL of a GY30 medium.

Moranoline-producing strains were selected by measuring alpha-glucosidase inhibition (AGI) activity in single colony culture solutions isolated from samples. For the measurement of AGI activity, 75 μL of a 0.1 M potassium phosphate buffer (pH 6.8) was added to 5 μL of a culture supernatant, and then 20 μL of an 0.8% (w/v) alpha-glucosidase enzyme (I1630, Sigma) thereto to react the resulting mixture at 37° C. for 5 minutes. Thereafter, 50 L of 12 mM 4-p-nitrophenyl-α-glucopyranoside (4-pNPG) was added as a substrate and the resulting mixture was reacted at 37° C. for 30 minutes. To terminate the reaction, 50 L of 0.2 M sodium carbonate was added, then absorbance was measured at 405 nm, and 100 strains with excellent AGI activity were selected.

To confirm the moranoline-producing ability of 100 strains with excellent AGI activity, flask culture was performed. Flask culture was performed using a 500 mL baffled flask including 100 mL of a GY50 medium (50 g/L glucose, 30 g/L yeast extract, 0.5 g/L KH₂PO₄, and 0.5 g/L (NH₄)₂SO₄) at a culture temperature of 37° C. and a stirring speed of 200 rpm for 72 hours. The amount of moranoline produced in the culture solution was analyzed after conversion to a derivative using FMOC-Cl. 100 μL of a 0.4 M potassium borate buffer (pH 8.5) was added to 100 μL of a culture supernatant and then mixed well. Thereafter, 200 μL of 5 mM FOMC-Cl was added thereto, the resulting mixture was mixed well, and then the mixture was allowed to react at 20° C. for 20 minutes. To terminate the reaction, 100 μL of 0.1 M glycine was added thereto, and then 500 μL of 1.9% (v/v) acetic acid was added thereto to stabilize the reaction solution. The final reaction solution was centrifuged at 12,000 rpm for 10 minutes, filtered through a 0.22 m filter, and then used for HPLC analysis.

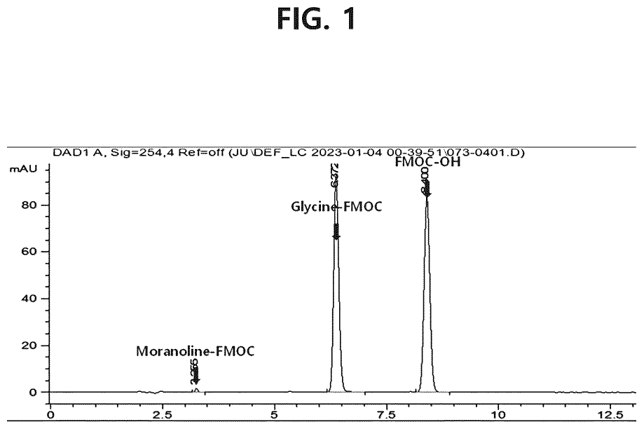

Moranoline-FOMC derivatives were analyzed using an HPLC system (Agilent 1200 series) equipped with a diode array detector (DAD). As a column used for the analysis, a Zorbax SBC18 column (4.6×150 mm, 5 m, Agilent) was used, and the analysis was performed under the following conditions: sample injection amount 10 μL, flow rate 0.8 mL/min, column oven temperature 30° C., mobile phase (acetonitrile:0.1% acetic acid=1:1), and detection wavelength 254 nm. As a result, four strains with excellent moranoline production were secured (FIG. 1).

The following Table 1 summarizes the amount of moranoline produced by the newly isolated strains.

TABLE 1

| Strain | Moranoline (mg/L) |
|---|---|
| #2-27 | 83.4 |
| #2-39 | 54.9 |
| #10-18 | 114.7 |
| #11-20 | 51.0 |

### 2) Selection of Excellent Novel Microorganism Producing Moranoline

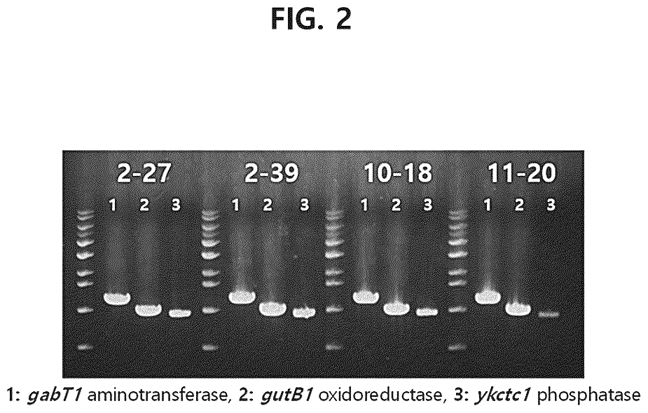

It was confirmed whether three enzymes involved in the biosynthesis of moranoline (GabT1: aminotransferase, YktC1: phosphatase, GutB1: oxidoreductase) were present in four novel microorganisms that produce moranoline. The primers for PCR reaction of each gene are shown in the following table. Genomic DNA was extracted from four novel moranoline-producing microorganisms and subjected to PCR. As a result, the genes gabT1, yktC1, and gutB1 involved in the biosynthesis of moranoline were confirmed in all four newly isolated microorganisms (FIG. 2).

TABLE 2
Primer sequences of genes related to
moranoline biosynthesis

| Gene | | Sequence |
|---|---|---|
| GabT1 | Forward | ATGGGAACGAAGGAAATCACGAATCCA |
| (1296 bp) | | (SEQ ID NO: 1) |
| | Reverse | TCACTTGATTTCCTCCAATAGCTTGCG |
| | | (SEQ ID NO: 2) |
| Yktc1 | Forward | GTGAGAGACTATATCATCGA |
| (951 bp) | | (SEQ ID NO: 3) |
| | Reverse | TTAGGAGTCCAGACCAACGCCTTCATA |
| | | (SEQ ID NO: 4) |
| GutB1 | Forward | ATGAAGGCGTTGGTCTGGACTCCTAAT |
| (1047 bp) | | (SEQ ID NO: 5) |
| | Reverse | TTATAAAAGTTTCGGATCAGAC |
| | | (SEQ ID NO: 6) |

In order to select microorganisms that produce a large amount of moranoline, the production of moranoline by four newly isolated microorganisms was analyzed depending on the culture time. The four newly isolated microorganisms were cultured in a 500 mL baffled flask including 100 mL of a GY50 medium (50 g/L glucose, 30 g/L yeast extract, 0.5 g/L KH₂PO₄, and 0.5 g/L (NH₄)₂SO₄) at a culture temperature of 37° C. and a stirring speed of 200 rpm for 96 hours. The amount of moranoline produced in the culture solution was analyzed using HPLC after conversion to a derivative using FMOC-Cl. As a result, it was confirmed that the 10-18 strain was the most effective in moranoline production over time.

TABLE 3

Moranoline (mg/L)

| Time (h) | #2-27 | #2-39 | #10-18 | #11-20 |
|---|---|---|---|---|
| 0 | 0.0 | 0.0 | 0.0 | 0.0 |
| 24 | 0.0 | 0.0 | 25.9 | 0.0 |
| 48 | 30.2 | 20.4 | 84.2 | 17.4 |
| 72 | 80.4 | 48.2 | 108.5 | 50.6 |
| 96 | 70.8 | 40.2 | 85.4 | 58.3 |

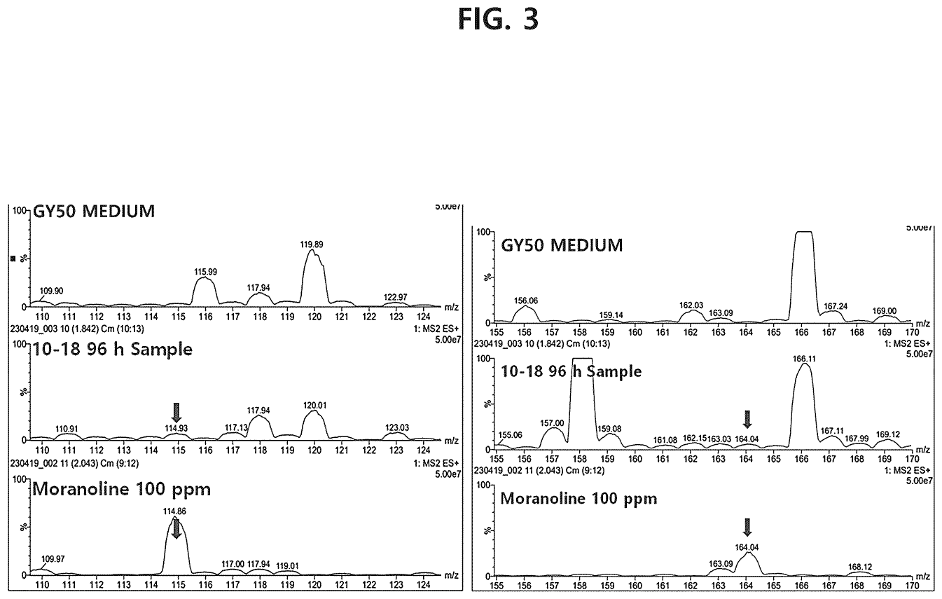

For the qualitative analysis of moranoline in the 96-hour culture solution of the 10-18 strain obtained through the aforementioned culture, LC-MSMSMS was used to analyze the presence or absence of moranoline in the culture solution. As a result of the analysis, a peak which is the same as the moranoline standard substance was confirmed in the 96-hour culture solution of the 10-18 strain, confirming that the newly isolated 10-18 strain produces moranoline (FIG. 3).

### 3) Classification and Nomenclature of Excellent Novel Microorganism Producing Moranoline

To identify the newly isolated microorganism 10-18 strain producing moranoline, a portion of the 16 rRNA sequence (SEQ TD NO: 7) was analyzed, and genetic identification was performed based on the analysis results.

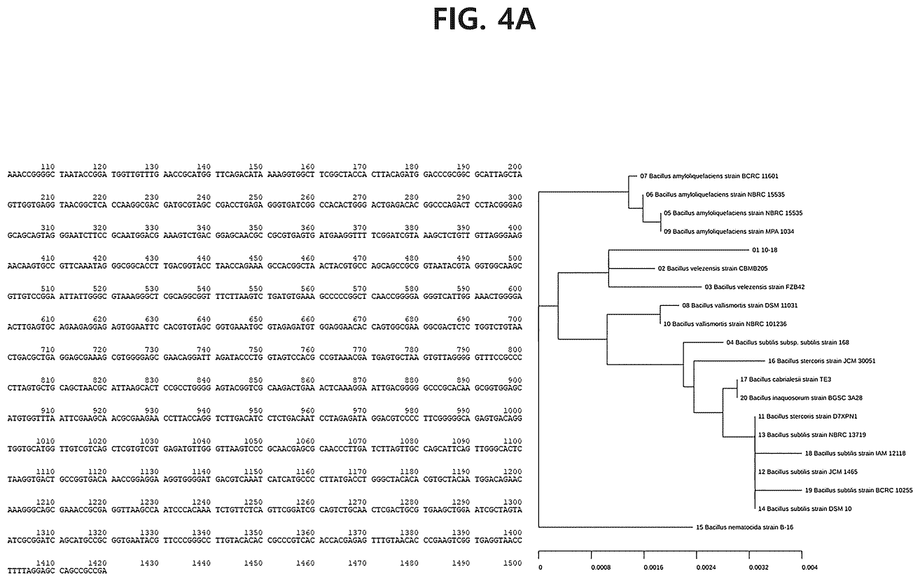

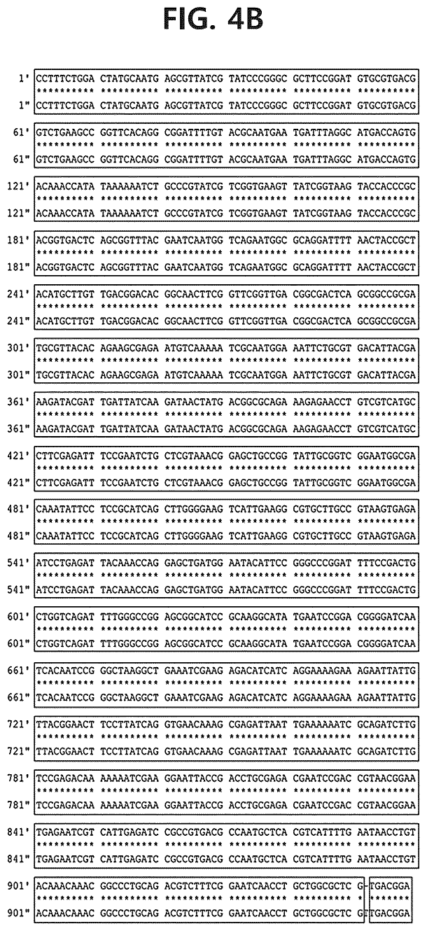

A portion of the 16S rRNA sequence of the newly isolated microorganism 10-18 strain is shown in the following Table 4, and the newly isolated microorganism 10-18 strain producing moranoline was classified as belonging to Bacillus velezensis (FIG. 4A).

TABLE 4
SEQ ID NO: 7
TGCAGTCGAGCGGACAGATGGGAGCTTGCTCCCTGATGTTAGCGGCGG
ACGGGTGAGTAACACGTGGGTAACCTGCCTGTAAGACTGGGATAACTC
CGGGAAACCGGGGCTAATACCGGATGGTTGTTTGAACCGCATGGTTCA
GACATAAAAGGTGGCTTCGGCTACCACTTACAGATGGACCCGCGGCGC
ATTAGCTAGTTGGTGAGGTAACGGCTCACCAAGGCGACGATGCGTAGC
CGACCTGAGAGGGTGATCGGCCACACTGGGACTGAGACACGGCCCAGA
CTCCTACGGGAGGCAGCAGTAGGGAATCTTCCGCAATGGACGAAAGTC
TGACGGAGCAACGCCGCGTGAGTGATGAAGGTTTTCGGATCGTAAAGC
TCTGTTGTTAGGGAAGAACAAGTGCCGTTCAAATAGGGCGGCACCTTG
ACGGTACCTAACCAGAAAGCCACGGCTAACTACGTGCCAGCAGCCGCG
GTAATACGTAGGTGGCAAGCGTTGTCCGGAATTATTGGGCGTAAAGGG
CTCGCAGGCGGTTTCTTAAGTCTGATGTGAAAGCCCCCGGCTCAACCG
GGGAGGGTCATTGGAAACTGGGGAACTTGAGTGCAGAAGAGGAGAGTG
GAATTCCACGTGTAGCGGTGAAATGCGTAGAGATGTGGAGGAACACCA
GTGGCGAAGGCGACTCTCTGGTCTGTAACTGACGCTGAGGAGCGAAAG
CGTGGGGAGCGAACAGGATTAGATACCCTGGTAGTCCACGCCGTAAAC
GATGAGTGCTAAGTGTTAGGGGGTTTCCGCCCCTTAGTGCTGCAGCTA
ACGCATTAAGCACTCCGCCTGGGGAGTACGGTCGCAAGACTGAAACTC
AAAGGAATTGACGGGGGCCCGCACAAGCGGTGGAGCATGTGGTTTAAT
TCGAAGCAACGCGAAGAACCTTACCAGGTCTTGACATCCTCTGACAAT
CCTAGAGATAGGACGTCCCCTTCGGGGGCAGAGTGACAGGTGGTGCAT
GGTTGTCGTCAGCTCGTGTCGTGAGATGTTGGGTTAAGTCCCGCAACG
AGCGCAACCCTTGATCTTAGTTGCCAGCATTCAGTTGGGCACTCTAAG
GTGACTGCCGGTGACAAACCGGAGGAAGGTGGGGATGACGTCAAATCA
TCATGCCCCTTATGACCTGGGCTACACACGTGCTACAATGGACAGAAC
AAAGGGCAGCGAAACCGCGAGGTTAAGCCAATCCCACAAATCTGTTCT
CAGTTCGGATCGCAGTCTGCAACTCGACTGCGTGAAGCTGGAATCGCT
AGTAATCGCGGATCAGCATGCCGCGGTGAATACGTTCCCGGGCCTTGT
ACACACCGCCCGTCACACCACGAGAGTTTGTAACACCCGAAGTCGGTG
AGGTAACCTTTTAGGAGCCAGCCGCCGA

In addition, to identify the 10-18 strain as a novel microorganism belonging to Bacillus velezensis, gyrA gene analysis of the 10-18 strain was performed. The primers for PCR reaction of the gyrA gene are shown in the following Table 5. A portion of the gyrA gene sequence (SEQ ID NO: 10) of the 10-18 strain subjected to the comparative analysis and a portion of the gyrA gene sequence (SEQ ID NO: 11) of the comparative strain Bacillus velezensis strain NY12-2 (depository institution: National Institute of Agricultural Science, Accession Number: KACC92193P) are shown in the following Table 6.

As a result of comparison, it was confirmed that the gene gyrA sequence of the 10-18 strain was different from the gene gyrA sequence of a known Bacillus velezensis strain NY12-2 (FIG. 4B). Therefore, the present inventors named the novel 10-18 strain Bacillus velezensis AmoreLumina.

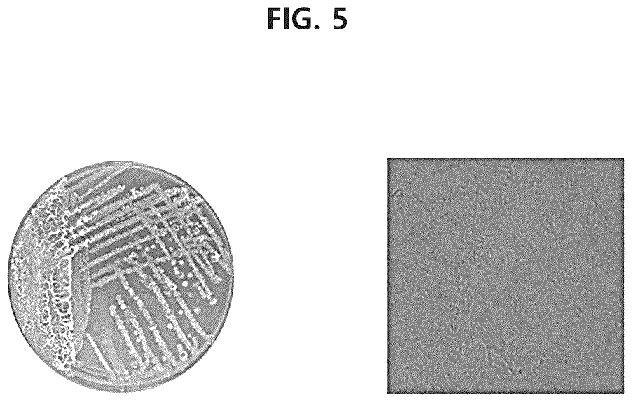

A colony photograph of colonies of the solid plate medium of a B. velezensis AmoreLumina strain, a novel microorganism producing moranoline, and an optical microscope photograph of the bacterial cells of the strain are shown in FIG. 5.

TABLE 5
Primer sequence of gyr A sequence

| Gene | | Sequence |
|---|---|---|
| gyrA | Forward | CAGTCAGGAAATGCGTACGTCCTT |
| | | (SEQ ID NO: 8) |
| | Reverse | CAAGGTAATGCTCCAGGCATTGCT |
| | | (SEQ ID NO: 9) |

TABLE 6

| | |
|---|---|
| SEQ ID NO: 10 | CCTTTCTGGACTATGCAATGAGCGTTATCGTATCCCGGGCGCTTCCGGATGTG |
| | CGTGACGGTCTGAAGCCGGTTCACAGGCGGATTTTGTACGCAATGAATGATT |
| | TAGGCATGACCAGTGACAAACCATATAAAAAATCTGCCCGTATCGTCGGTGA |
| | AGTTATCGGTAAGTACCACCCGCACGGTGACTCAGCGGTTTACGAATCAATG |
| | GTCAGAATGGCGCAGGATTTTAACTACCGCTACATGCTTGTTGACGGACACG |
| | GCAACTTCGGTTCGGTTGACGGCGACTCAGCGGCCGCGATGCGTTACACAG |
| | AAGCGAGAATGTCAAAAATCGCAATGGAAATTCTGCGTGACATTACGAAAG |
| | ATACGATTGATTATCAAGATAACTATGACGGCGCAGAAAGAGAACCTGTCGT |
| | CATGCCTTCGAGATTTCCGAATCTGCTCGTAAACGGAGCTGCCGGTATTGCG |
| | GTCGGAATGGCGACAAATATTCCTCCGCATCAGCTTGGGGAAGTCATTGAAG |
| | GCGTGCTTGCCGTAAGTGAGAATCCTGAGATTACAAACCAGGAGCTGATGG |
| | AATACATTCCGGGCCCGGATTTTCCGACTGCTGGTCAGATTTTGGGCCGGAG |
| | CGGCATCCGCAAGGCATATGAATCCGGACGGGGATCAATCACAATCCGGGCT |
| | AAGGCTGAAATCGAAGAGACATCATCAGGAAAAGAAAGAATTATTGTTACG |
| | GAACTTCCTTATCAGGTGAACAAAGCGAGATTAATTGAAAAAATCGCAGATC |
| | TTGTCCGAGACAAAAAAATCGAAGGAATTACCGACCTGCGAGACGAATCCG |
| | ACCGTAACGGAATGAGAATCGTCATTGAGATCCGCCGTGACGCCAATGCTCA |
| | CGTCATTTTGAATAACCTGTACAAACAAACGGCCCTGCAGACGTCTTTCGGA |
| | ATCAACCTGCTGGCGCTCGTGACGGA |
| SEQ ID NO: 11 | CCTTTCTGGACTATGCAATGAGCGTTATCGTATCCCGGGCGCTTCCGGATGTG |
| | CGTGACGGTCTGAAGCCGGTTCACAGGCGGATTTTGTACGCAATGAATGATT |
| | TAGGCATGACCAGTGACAAACCATATAAAAAATCTGCCCGTATCGTCGGTGA |
| | AGTTATCGGTAAGTACCACCCGCACGGTGACTCAGCGGTTTACGAATCAATG |
| | GTCAGAATGGCGCAGGATTTTAACTACCGCTACATGCTTGTTGACGGACACG |
| | GCAACTTCGGTTCGGTTGACGGCGACTCAGCGGCCGCGATGCGTTACACAG |
| | AAGCGAGAATGTCAAAAATCGCAATGGAAATTCTGCGTGACATTACGAAAG |
| | ATACGATTGATTATCAAGATAACTATGACGGCGCAGAAAGAGAACCTGTCGT |
| | CATGCCTTCGAGATTTCCGAATCTGCTCGTAAACGGAGCTGCCGGTATTGCG |
| | GTCGGAATGGCGACAAATATTCCTCCGCATCAGCTTGGGGAAGTCATTGAAG |
| | GCGTGCTTGCCGTAAGTGAGAATCCTGAGATTACAAACCAGGAGCTGATGG |
| | AATACATTCCGGGCCCGGATTTTCCGACTGCTGGTCAGATTTTGGGCCGGAG |
| | CGGCATCCGCAAGGCATATGAATCCGGACGGGGATCAATCACAATCCGGGCT |
| | AAGGCTGAAATCGAAGAGACATCATCAGGAAAAGAAAGAATTATTGTTACG |
| | GAACTTCCTTATCAGGTGAACAAAGCGAGATTAATTGAAAAAATCGCAGATC |
| | TTGTCCGAGACAAAAAAATCGAAGGAATTACCGACCTGCGAGACGAATCCG |
| | ACCGTAACGGAATGAGAATCGTCATTGAGATCCGCCGTGACGCCAATGCTCA |
| | CGTCATTTTGAATAACCTGTACAAACAAACGGCCCTGCAGACGTCTTTCGGA |
| | ATCAACCTGCTGGCGCTCGTTGACGGA |

2. Development of Moranoline Production Process by B. velezensis AmoreLumina Strain

### 1) Effect of Initial pH

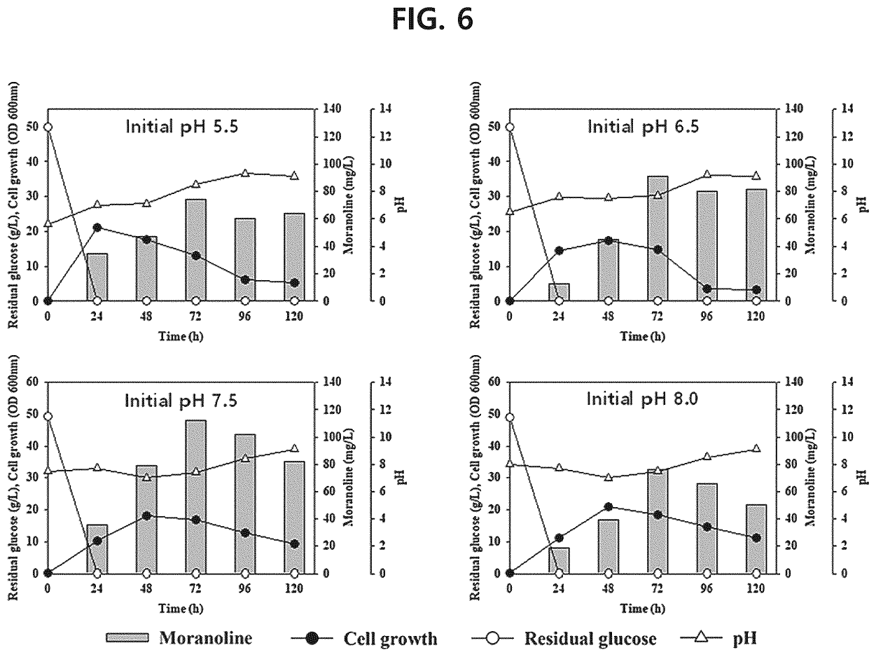

The effect of initial culture pH on the production of moranoline by the B. velezensis AmoreLumina strain was confirmed by flask culture. Flask culture was performed using a 500 mL baffled flask including 100 mL of a GY50 medium under the initial culture pH condition of 5.5 to 8.5. The culture was performed under the culture conditions of a culture temperature of 37° C. and a stirring speed of 200 rpm for 120 hours. Samples were collected every 24 hours and used for analysis, and the amount of moranoline produced in the culture solution was analyzed by HPLC after conversion to a derivative using FMOC-Cl. The bacterial cell growth was analyzed by measuring the absorbance at 600 nm using a spectrophotometer, and the residual substrate was analyzed using HPLC. As a result, the carbon source in the culture solution was exhausted at 24 hours of culture under all culture conditions, but the highest amount of moranoline was produced at 72 hours of culture under the initial culture pH condition of 7.5. Under an initial culture pH condition of 7.5, the amount of moranoline produced decreased and the pH increased after 72 hours of culture (FIG. 6).

### 2) Production of Moranoline Depending on Type of Carbon Source

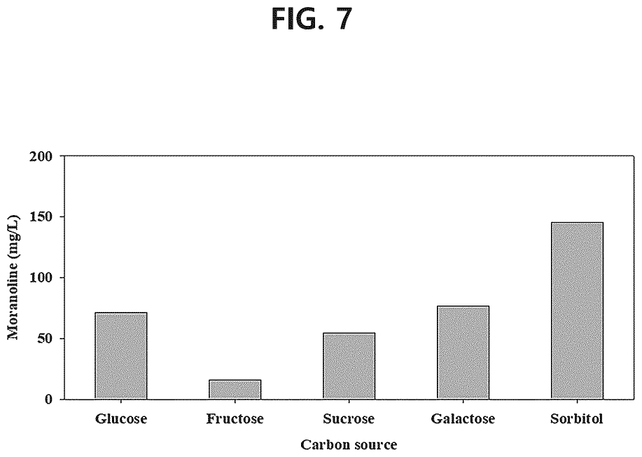

The effect of the type of carbon source on the production of moranoline by the B. velezensis AmoreLumina strain was confirmed by flask culture. Flask culture was performed using a 500 mL baffled flask including 100 mL of a medium, and in the composition of the culture medium, 50 g/L carbon source, 30 g/L yeast extract, 0.5 g/L KH₂PO₄, and 0.5 g/L (NH₄)₂SO₄ were used, and glucose, fructose, sucrose, galactose, and sorbitol were used as carbon sources. The culture was performed under the culture conditions of a culture temperature of 37° C., a stirring speed of 200 rpm, and an initial pH of 7.5 for 72 hours. Samples were collected every 24 hours and used for analysis, and the amount of moranoline produced in the culture solution was analyzed by HPLC after conversion to a derivative using FMOC-Cl. As a result, the highest amount of moranoline was produced when sorbitol was used as the carbon source (FIG. 7).

### 3) Production of Moranoline Depending on Type of Nitrogen Source

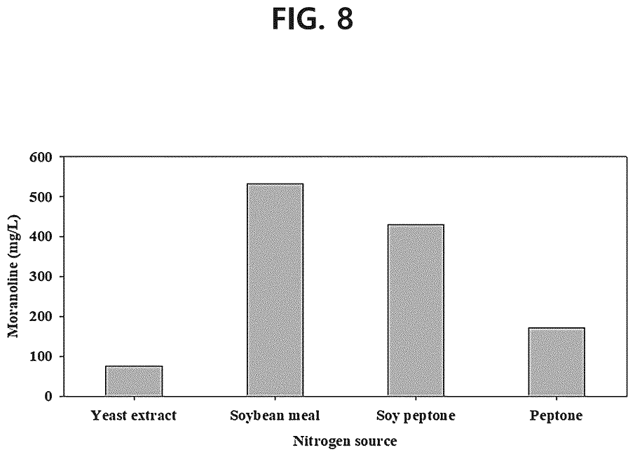

The effect of the type of nitrogen source on the production of moranoline by the B. velezensis AmoreLumina strain was confirmed by flask culture. Flask culture was performed using a 500 mL baffled flask including 100 mL of a medium, and in the composition of the culture medium, 30 g/L nitrogen source, 0.5 g/L KH₂PO₄, and 0.5 g/L (NH₄)₂SO₄ were used, and a yeast extract, soybean meal, soy peptone, and peptone were used as nitrogen sources. The culture was performed under the culture conditions of a culture temperature of 37° C., a stirring speed of 200 rpm, and an initial pH of 7.5 for 72 hours. Samples were collected every 24 hours and used for analysis, and the amount of moranoline produced in the culture solution was analyzed by HPLC after conversion to a derivative using FMOC-Cl. As a result, the highest amount of moranoline was produced when soybean meal, an inexpensive nitrogen source, was used as the nitrogen source (FIG. 8).

### 4) Production of Moranoline Depending on Concentration of Carbon Source

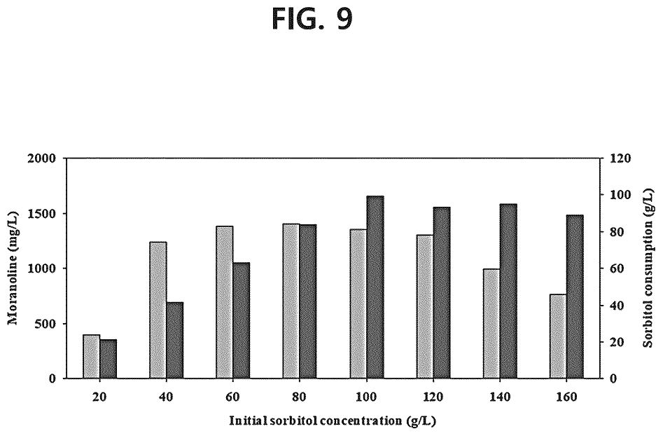

The effect of the concentration of carbon source on the production of moranoline by the B. velezensis AmoreLumina strain was confirmed by flask culture. Flask culture was performed using a 500 mL baffled flask including 100 mL of a medium, and the culture medium was used by adding sorbitol at each concentration (20 to 180 g/L) to 30 g/L soybean meal, 0.5 g/L KH₂PO₄, and 0.5 g/L (NH₄)₂SO₄. The culture was performed under the culture conditions of a culture temperature of 37° C., a stirring speed of 200 rpm, and an initial pH of 7.5 for 96 hours. Samples were collected every 24 hours and used for analysis, and the amount of moranoline produced in the culture solution was analyzed by HPLC after conversion to a derivative using FMOC-Cl. The results of culturing the B. velezensis AmoreLumina strain for 48 hours depending on the initial sorbitol concentration are shown in FIG. 9. There was no significant difference in the production of moranoline at initial sorbitol concentrations of 60 g/L to 100 g/L, but it was confirmed that the amount of moranoline produced decreased when the initial sorbitol concentration was 40 g/L or less, and that the amount of moranoline produced decreased and the amount substrate consumed also decreased when the initial sorbitol concentration was 120 g/L or more (FIG. 9).

### 5) Production of Moranoline Depending on Concentration of Nitrogen Source

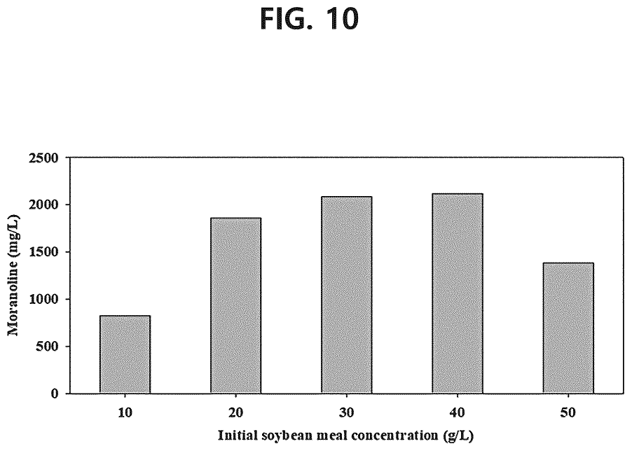

The effect of the concentration of nitrogen source on the production of moranoline by the B. velezensis AmoreLumina strain was confirmed by flask culture. Flask culture was performed using a 500 mL baffled flask including 100 mL of a medium, and the culture medium was used by adding soybean meal at each concentration (10 to 50 g/L) to 60 g/L sorbitol, 0.5 g/L KH₂PO₄, and 0.5 g/L (NH₄)₂SO₄. The culture was performed under the culture conditions of a culture temperature of 37° C., a stirring speed of 200 rpm, and an initial pH of 7.5 for 96 hours. Samples were collected every 24 hours and used for analysis, and the amount of moranoline produced in the culture solution was analyzed by HPLC after conversion to a derivative using FMOC-Cl. The results of culturing the B. velezensis AmoreLumina strain for 72 hours depending on the initial soybean meal concentration are shown in FIG. 10. There was no significant difference in the production of moranoline at initial soybean meal concentrations of 30 g/L to 40 g/L, but the amount of moranoline produced decreased when the initial soybean meal concentration was 20 g/L or less, and the amount of moranoline produced decreased when the initial soybean meal concentration was 50 g/L or more (FIG. 10).

6) Culture of B. velezensis AmoreLumina Strain Using Optimal Medium for Producing Moranoline

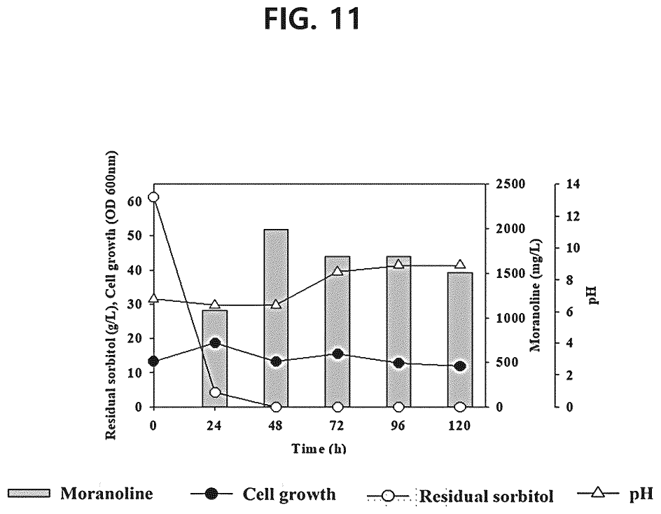

Using the established carbon and nitrogen sources, flask culture of the B. velezensis AmoreLumina strain was performed for producing moranoline. Flask culture was performed using a 500 mL baffled flask including 100 mL of SM60 medium (60 g/L sorbitol, 30 g/L soybean meal, 0.5 g/L KH₂PO₄, and 0.5 g/L (NH₄)₂SO₄). The culture was performed under the culture conditions of a culture temperature of 37° C., a stirring speed of 200 rpm, and an initial pH of 7.5 for 120 hours. Samples were collected every 24 hours and used for analysis, and the amount of moranoline produced in the culture solution was analyzed by HPLC after conversion to a derivative using FMOC-Cl. The bacterial cell growth was analyzed by measuring the absorbance at 600 nm using a spectrophotometer, and the residual substrate was analyzed using HPLC. As a result, using a medium using a single carbon source sorbitol and a nitrogen source soybean meal as a substrate, the B. velezensis AmoreLumina strain produced 1,995 mg/L of moranoline within 48 hours of culture (FIG. 11).

3. Scale Up (5 L) of Moranoline Production Process by B. velezensis AmoreLumina Strain

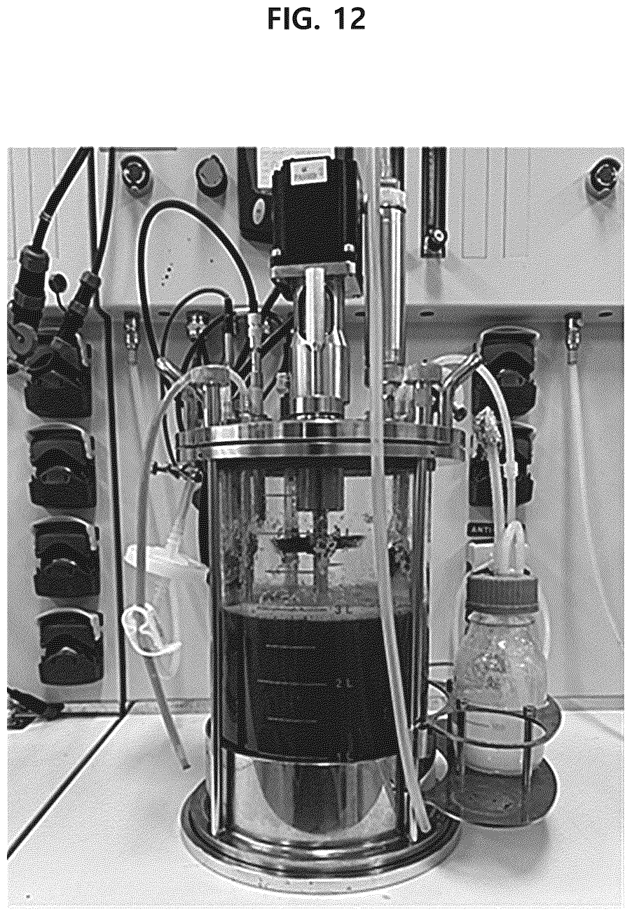

Scale-up is essential for the mass production of high-concentration moranoline. Therefore, a high-concentration moranoline production process was established using the B. velezensis AmoreLumina strain using a 5 L fermenter (FIG. 12).

### 1) Production of Moranoline Depending on Stirring Speed

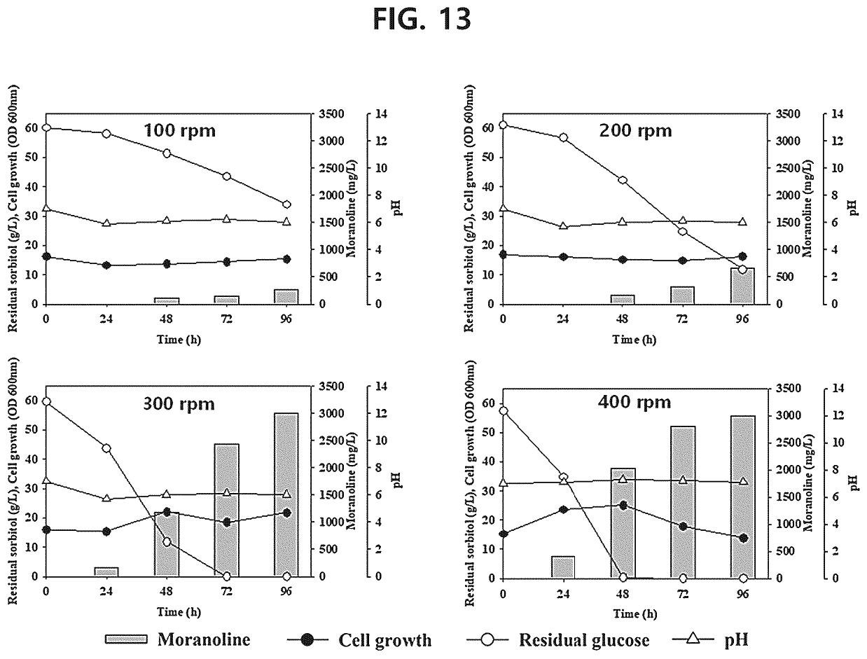

The effect of stirring speed on the production of moranoline by the B. velezensis AmoreLumina strain was confirmed by batch culture using a 5 L fermenter. Batch culture was performed using a 5 L fermenter (CNS) including 3 L of SM60 medium. The batch culture was performed under the culture conditions of an inoculum amount of 1% (v/v), a culture temperature of 37° C., and an initial pH of 7.0 for 96 hours. The batch culture was performed under the condition of a stirring speed of 100 rpm to 400 rpm. Samples were collected every 24 hours and used for analysis, and the amount of moranoline produced in the culture solution was analyzed by HPLC after conversion to a derivative using FMOC-Cl. The bacterial cell growth was analyzed by measuring the absorbance at 600 nm using a spectrophotometer, and the residual substrate was analyzed using HPLC. As a result, 2998.3 mg/L and 3005.5 mg/L of moranoline were produced for 96 hours of batch culture under the condition of a stirring speed of 300 rpm and 400 rpm, respectively (FIG. 13).

An LC-MSMS analysis was commissioned for quantitative analysis of the culture solution of the B. velezensis AmoreLumina strain cultured using a fermenter. An LC-MSMS analysis of a culture solution containing 3005.5 mg/L moranoline, which had been analyzed through derivatization, was commissioned to the Center for Industrialization of Agricultural and Livestock Microorganisms. As a result, quantitative analysis revealed that the B. velezensis AmoreLumina strain culture solution contained 2434.7 mg/L moranoline.

Table 7 compares the amounts of moranoline produced by the strain herein and by microorganisms reported to date.

TABLE 7

| | | | Carbon | Nitrogen | | | |
|---|---|---|---|---|---|---|---|
| | 1-DNJ | Fermentation | source | source | Fermentation | Fermentation | |
| Strain | (mg/L) | time (h) | (g/L) | (g/L) | Scale | Type | Year |
| B. velezensis | 2434.7 | 96 | Sorbitol | Soybean | 3 L/5 L | Batch | |
| AmoreLumina | | | (50) | meal (30) | fermenter | | |
| Bacillus | 1632.5 | 96 | Glucose | Peptone | 1.8 L/3 L | Fed-Batch | 2023 |
| amyloliquefaciens | | | (40) | (20) Yeast | fermenter | | |
| (GMO) | | | | extract (15) | | | |
| B. subtilis MORI | 824 | 120 | Galactose | Soybean | 1 L/2.5 L | Batch | 2021 |
| | | | (43) | meal (32) | fermenter | | |
| B. subtilis MORI | 600 | 120 | Galactose | Soybean | 30 L/50 L | Batch | 2021 |
| | | | (43) | meal (32) | fermenter | | |
| B. | 1140 | 120 | Lactose | Ammonium | 100 mL/500 | Batch | 2017 |
| amyloliquefaciens | | | (25) | sulfate (4) | mL flask | | |
| B. | 460 | 120 | Sorbitol | Soybean | 50 mL/500 | Batch | 2013 |
| amyloliquefaciens | | | (50) | peptone | mL flask | | |
| AS385 | | | | (40) | | | |
| S. lavendulae | 42.8 | 264 | Soluble | Tryptone | — | — | 2011 |
| | | | starch (80) | (10) | | | |
| B. subtilis S10 | 750 | 48 | Galactose | Polypeptone | 3 L/5 L | Batch | 2008 |
| | | | (10) | (16) | fermenter | | |
| Streptomyces sp. | 640 | 120 | Lactose | Soybean | 3 L/6.6 L | Batch | 1997 |
| | | | (25) | meal (20) | fermenter | | |

4. Isolation and Purification of Moranoline in B. velezensis AmoreLumina Strain Culture Solution

The B. velezensis AmoreLumina strain culture solution was scaled up in a 5 L fermenter, and a culture solution with the highest amount of moranoline produced (about 2.4 g/L) was used to perform a moranoline isolation and purification test.

The purity of moranoline in the culture solution was measured by a high-performance liquid chromatography (HPLC) method. For quantitative analysis, moranoline (Sigma, ≥95.0% (HPLC)) was dissolved in distilled water to a concentration of 1000 ppm, 500 ppm, 250 ppm, 125 ppm, and 62.5 ppm (w/v), respectively, and then diluted to ½ in a mixture of acetonitrile and distilled water (50:50, v/v, containing 6.5 mM ammonium acetate, pH 5.5) and measured. After the culture solution was diluted to ½ in a mixture of acetonitrile and distilled water (50:50, v/v, containing 6.5 mM ammonium acetate, pH 5.5), the content of moranoline in the culture solution was measured.

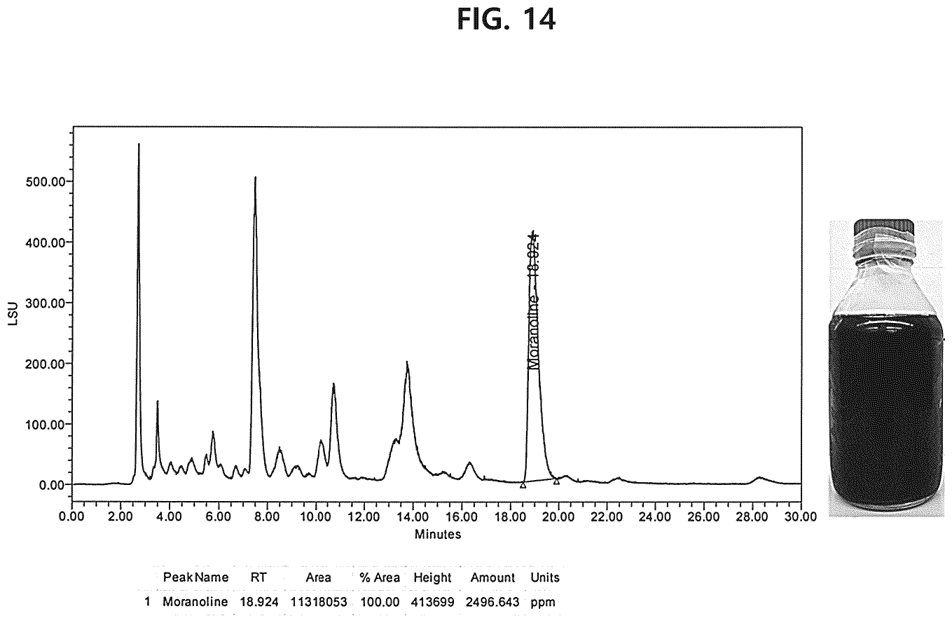

HPLC analysis was performed based on a method using an amide type column and an evaporative light scattering detector (ELSD). As a column used for the analysis, a TSK-Gel-amide-80 column (4.6 mm×250 mm, 5 m, Tosoh, Tokyo, Japan) was used, and the sample injection amount, the flow rate, and the column temperature were set to 10 μl, 1 mL/min, and 60° C., respectively. Isolation was performed using a mixture of acetonitrile and distilled water (81:19, v/v, containing 6.5 mM ammonium acetate, pH 5.5). As a result, the content of moranoline in the culture solution was about 2.49 g/L, and was confirmed as a proportion of about 21% of the total culture solution components (FIG. 14).

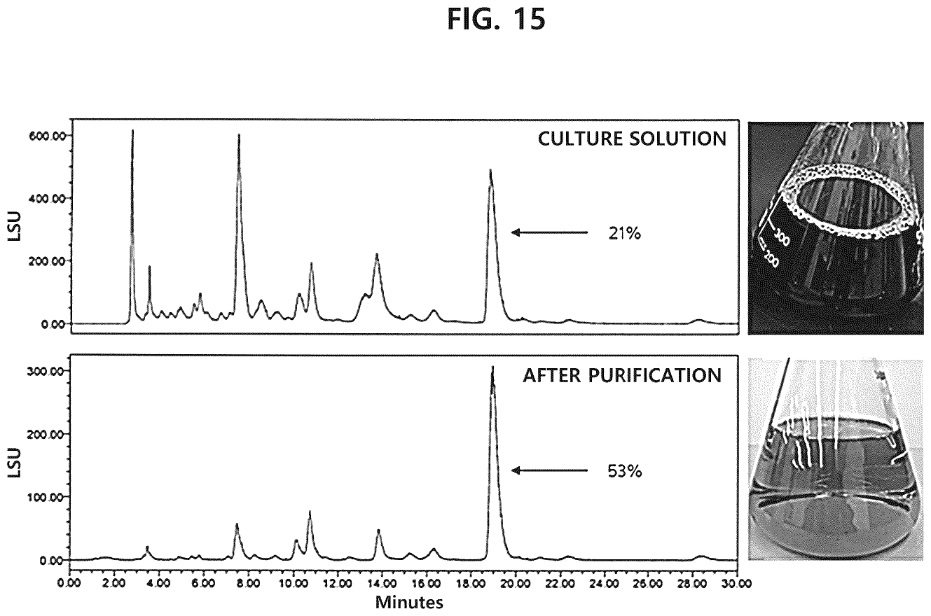

The B. velezensis AmoreLumina strain culture solution had a dark brown color and a distinctive unpleasant odor. In addition, since the proportion of moranoline in the culture solution was about 21%, a purification process was performed to increase the purity of moranoline. The pH was adjusted to 3.0 by adding citric acid crystals to 1 L of the culture solution. The solution was centrifuged at 7000 rpm for 10 minutes, and the supernatant was collected and filtered through a 0.2 m filter. Thereafter, the filtrate was applied to a glass column packed with 200 g of an ion exchange resin (HP-20), and then an effluent was obtained at a rate of 20 mL/min. Thereafter, the effluent was passed through a cation exchange resin (AmberLyst-15WET H⁺ form, 200 g) at a rate of 20 ml/min to adsorb an active portion including moranoline, and the active portion was washed with about 5 L of distilled water until the pH of the effluent reached 6 to 7, and then was eluted at a rate of 20 ml/min using 0.5 N H₄OH. The effluent was fractionated into 500 ml portions, and only a first fraction, which had the highest concentration of moranoline, was taken, concentrated under reduced pressure, and freeze-dried (obtaining about 4.6 g of a dry powder). After the freeze-dried culture solution extract was dissolved in a mixture of acetonitrile and distilled water (50:50, v/v, containing 6.5 mM ammonium acetate, pH 5.5) at a concentration of 5 mg/ml, the purity of moranoline in the culture solution extract was confirmed using HPLC. As a result, it was possible to obtain a culture solution extract in which the proportion of moranoline in the culture solution increased from 21% to 53% by about 2.5-fold or more. Further, it was confirmed that the culture solution having a transparent brown color and a significantly reduced distinctive unpleasant odor was obtained (FIG. 15).

5. Efficacy Evaluation of B. velezensis AmoreLumina Strain Culture Solution Extract
1) Confirmation of Alpha-Glucosidase Inhibition (AGI) Activity of B. velezensis AmoreLumina Strain Culture Solution

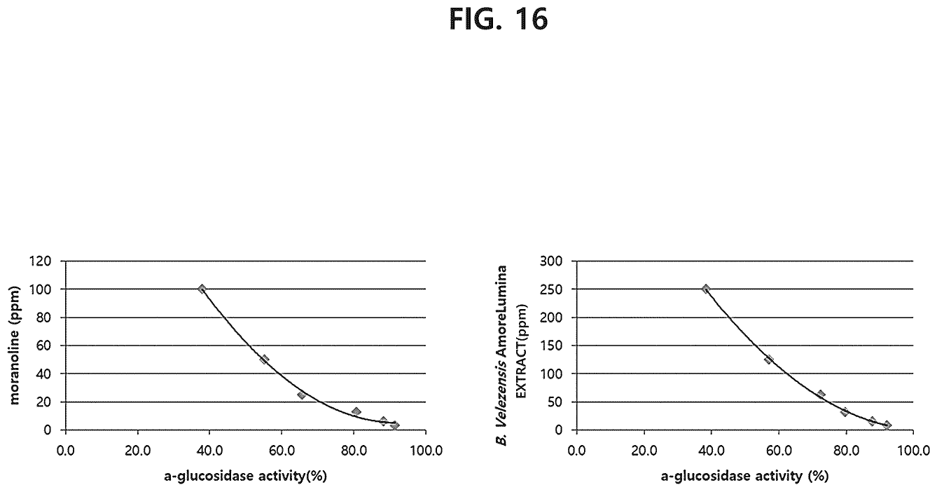

The AGI activity of a culture solution extract obtained through the aforementioned process was measured. To measure IC₅₀, the culture solution extract and a moranoline standard material were dissolved in a 0.1 M potassium phosphate buffer (pH 6.8) at each different concentration, and then the AGI activity was measured. For the measurement method, an AGI activity evaluation method used in the strain selection process was used. As a result, the IC₅₀ for moranoline was 62.3 ppm, and the IC₅₀ for the B. velezensis AmoreLumina strain culture solution extract (moranoline: about 20%) was confirmed to be 168.3 ppm, confirming that the extract had the IC₅₀ value at a concentration about 2.5-fold that of the moranoline standard material (FIG. 16).

2) Evaluation of Melanin Production Inhibitory Efficacy of B. velezensis AmoreLumina Strain Culture Solution Extract

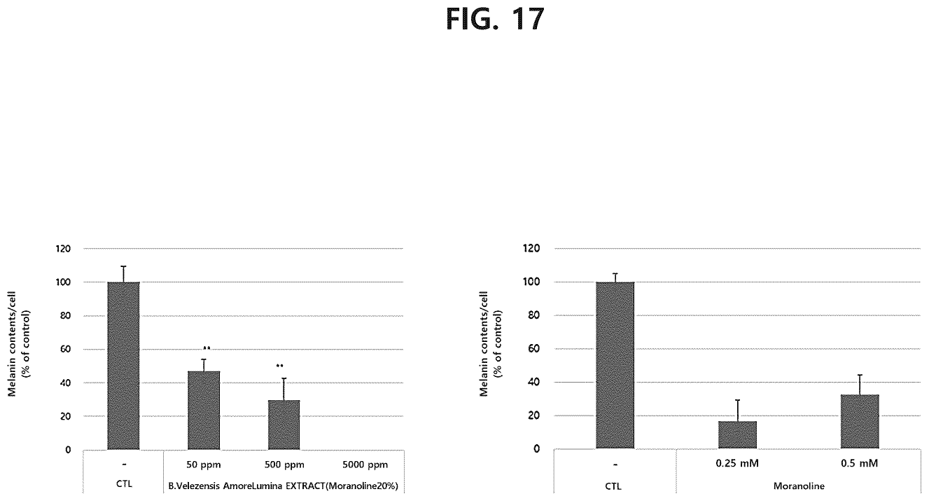

To confirm the melanin production inhibitory efficacy of the culture solution extract, normal human epidermal melanocytes (NHEMs, Cascade Biologics, Portland, OR, USA) isolated from normal human skin were prepared. The NHEMs were maintained in M-254 medium supplemented with human melanocyte growth supplements (HMGS) (Cascade Biologics, Inc., Mansfield, UK). After the freeze-dried extract was dissolved in the M-254 medium at a concentration of 50, 500, and 5,000 ppm (w/v), respectively, the NHEMs were treated with the dissolved extract. For comparison, 250 mM and 500 mM moranoline standard materials were dissolved in the M-254 medium at a concentration of 250 mM and 500 mM, respectively, and then the NHEMs were treated with the moranoline standard material. Thereafter, NHEM cells were cultured in a 37° C., 5% CO₂ incubator for up to 7 days, while changing the culture medium containing an experimental material every two days. After 7 days, the cells were treated with trypsin/EDTA, then centrifuged at 1000×g for 5 minutes, and washed twice with PBS, and a cell pellet was dissolved in 1 N NaOH. The homogenized cell extract was transferred to a 96-well plate, and a relative melanin amount was measured at an absorbance of 405 nm using an ELISA plate reader. As a result, it was confirmed that melanin synthesis was suppressed by 60% or more at 250 M to 500 M (40.79 ppm to 81.59 ppm) of moranoline, and the same efficacy was confirmed at 50 ppm to 500 ppm in the case of the B. velezensis 10-18 strain culture solution extract (moranoline about 20%) (FIG. 17).

3) Evaluation of Brightening Efficacy of B. velezensis AmoreLumina Strain Culture Solution in Artificial Skin

The brightening efficacy of the AmoreLumina strain culture solution extract was confirmed using a pigmented artificial skin model (MEL-300-B, MatTek, USA). The artificial skin was placed in Dulbecco's Modified Eagle's Medium (Lonza, Basel, Switzerland) culture medium containing 10% fetal bovine serum and 1% penicillin-streptomycin (Life Technologies, Grand Island, NY, USA), and treated by adding kojic acid (Sigma Aldrich, St. Louis, USA) as a positive control at a concentration of 7,000 g/mL and the AmoreLumina strain culture solution extract at a concentration of each of 50 g/mL, 500 g/mL, and 5,000 g/mL to the culture medium. The artificial skin was grown in a 37° C., 5% CO₂ incubator for a total of 14 days while changing the culture medium containing kojic acid or the strain culture solution extract every two days. After photographs of the artificial skin were taken with a digital camera (EOS760D, Cannon) on M (Manual exposure) mode, 1/60s, F6.3, ISO400, and manual focus adjustment at the beginning and end of the experiment, the RGB values of the photographs were obtained (Image-Pro Plus, Media Cybernetics Inc., US) and then the RGB values were converted to L (Lightness) values, which represent the lightness of the skin, using the Convert Rgb to Lab program (http://colormine.org/convert/rgb-to-lab). The variation in relative skin lightness during the evaluation period was obtained by the following equation, with a larger variation in relative skin lightness indicating a lighter skin color.

$$Variation in relative skin lightness = Δ L - Δ Lc \qquad [ Equation ]$$

In this case, ΔL is the difference between the L value on the final day of treatment (day 14) of the sample-treated group and the L value on the beginning day of sample treatment (day 0), that is, the variation in skin lightness of the skin treated with the sample for 14 days, and ΔLc is the variation in skin lightness of the control skin for 14 days.

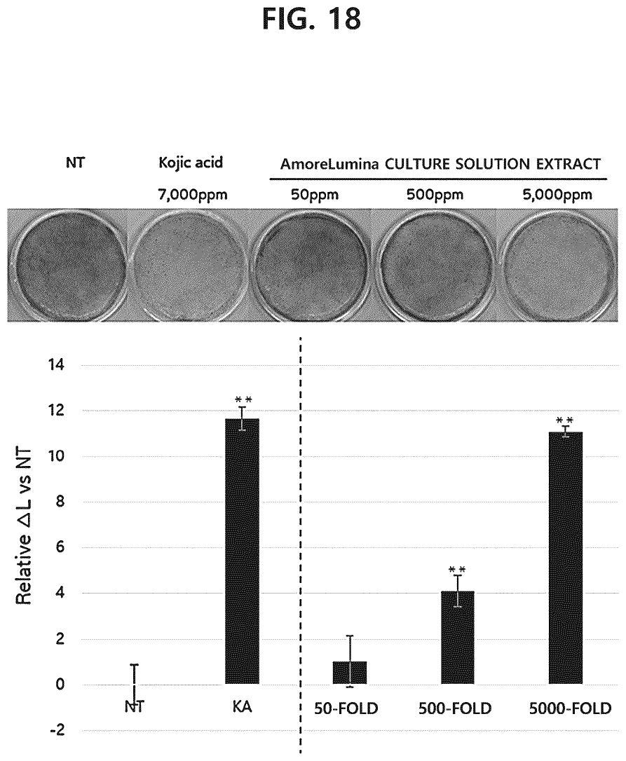

As a result of the experiment, it could be confirmed that as the concentration of the AmoreLumina strain culture solution extract increased, pigmentation was suppressed in the epidermis layer of the artificial skin, and thus, the variation in relative skin lightness increased to the level of kojic acid (FIG. 18).

### [Accession Number]

Name of depository institution: Korea Research Institute of Bioscience and Biotechnology, Korean Collection for Type Cultures

Accession Number: KCTC 15911BP

Date of deposit: May 20, 2024

[National research and development project supporting the present invention]
[Task serial number]1711196162
[Task number] KGM5482322
[Government department] Ministry of Science and ICT
[Task management (specialized) institute] Korea Research Institute of Bioscience and Biotechnology
[Research project] Korea Research Institute of Bioscience and Biotechnology Research Operational Expenses Support (Main Project Expenses)
[Research task] Development and commercialization of innovative materials for well-aging based on agricultural life microbiome
[Contribution ratio]1/1
[Task performing institute] Korea Research Institute of Bioscience and Biotechnology
[Research period] Jan. 1, 2024-Dec. 31, 2024

We claim:

1. A Bacillus velezensis AmoreLumina strain deposited under Accession Number KCTC 15911BP.

2. The strain of claim 1, wherein the strain has DNA encoding a gyrA gene comprising a sequence of SEQ ID NO: 10.

3. A method for producing moranoline, the method comprising culturing a composition comprising: the strain of claim 1; a lysate thereof, a culture solution thereof, or an extract of the culture solution as an active ingredient.

4. The method of claim 3, wherein the active ingredient suppresses a production of α-glucosidase.

5. A method for brightening the skin, the method comprising administering, to a subject in need of skin brightening, a composition comprising: the strain of claim 1; a lysate thereof; a culture solution thereof; or an extract of the culture solution as an active ingredient.

6. The method of claim 5, wherein the active ingredient is a culture solution extract of the strain, and the extract is used in a daily dosage of 10 mg/kg to 25,000 mg/kg.

7. The method of claim 5, wherein the brightening suppresses a production of melanin.

8. The method of claim 5, wherein the composition is a cosmetic or food composition.

9. The method of claim 5, wherein the brightening is the prevention or treatment of a hyperpigmentation disorder.

10. A medium composition for culturing a Bacillus velezensis strain, comprising sorbitol as a carbon source and soybean meal as a nitrogen source.

11. The composition of claim 10, wherein a pH of the composition is 5 to 10.

12. The composition of claim 10, wherein the sorbitol is present in an amount of more than 40 g/L and less than 120 g/L based on a total volume of the composition, or
the soybean meal is present in an amount of more than 20 g/L and less than 50 g/L based on the total volume of the composition.

13. The composition of claim 10, wherein the medium composition comprises 60 g/L to 100 g/L sorbitol, 30 g/L to 40 g/L soybean meal, 0.1 g/L to 1.0 g/L KH₂PO₄, and 0.1 g/L to 1.0 g/L (NH₄)₂SO₄.

14. The composition of claim 10, wherein the medium is for producing moranoline.

15. The composition of claim 10, wherein the strain is a strain deposited under Accession Number KCTC 15911BP.

16. A method for producing moranoline, the method comprising culturing a Bacillus velezensis strain, wherein the step is performed with stirring at a speed of 250 rpm to 450 rpm.

17. The method of claim 16, further comprising adjusting the pH of a strain culture solution obtained after the culture to 4 or less, and filtering the strain culture solution to purify moranoline.

18. The method of claim 17, further comprising adjusting the pH of an eluate from the filtered filtrate to a pH of 5.5 to 7.5 and fractionating the eluate.

19. The method of claim 16, wherein the culturing step is performed under a condition of a pH of 6.5 to 8.5 for 40 hours or more.

20. The method of claim 18, wherein the strain is a strain deposited under Accession Number KCTC 15911BP.


SEQUENCE LISTING (11 sequences)

SEQ ID NO: 1 - DNA, 27 bp, synthetic construct
  source 1..27: mol_type = other DNA
atgggaacgaaggaaatcacgaatcca

SEQ ID NO: 2 - DNA, 27 bp, synthetic construct
  source 1..27: mol_type = other DNA
tcacttgatttcctccaatagcttgcg

SEQ ID NO: 3 - DNA, 20 bp, synthetic construct
  source 1..20: mol_type = other DNA
gtgagagactatatcatcga

SEQ ID NO: 4 - DNA, 27 bp, synthetic construct
  source 1..27: mol_type = other DNA
ttaggagtccagaccaacgccttcata

SEQ ID NO: 5 - DNA, 27 bp, synthetic construct
  source 1..27: mol_type = other DNA
atgaaggcgttggtctggactcctaat

SEQ ID NO: 6 - DNA, 22 bp, synthetic construct
  source 1..22: mol_type = other DNA
ttataaaagtttcggatcagac

SEQ ID NO: 7 - RNA, 1420 nt, Bacillus velezensis
  source 1..1420: mol_type = rRNA
tgcagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccga

SEQ ID NO: 8 - DNA, 24 bp, synthetic construct
  source 1..24: mol_type = other DNA
cagtcaggaaatgcgtacgtcctt

SEQ ID NO: 9 - DNA, 24 bp, synthetic construct
  source 1..24: mol_type = other DNA
caaggtaatgctccaggcattgct

SEQ ID NO: 10 - DNA, 958 bp, Bacillus velezensis
  source 1..958: mol_type = genomic DNA
cctttctggactatgcaatgagcgttatcgtatcccgggcgcttccggatgtgcgtgacggtctgaagccggttcacaggcggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagatacgattgattatcaagataactatgacggcgcagaaagagaacctgtcgtcatgccttcgagatttccgaatctgctcgtaaacggagctgccggtattgcggtcggaatggcgacaaatattcctccgcatcagcttggggaagtcattgaaggcgtgcttgccgtaagtgagaatcctgagattacaaaccaggagctgatggaatacattccgggcccggattttccgactgctggtcagattttgggccggagcggcatccgcaaggcatatgaatccggacggggatcaatcacaatccgggctaaggctgaaatcgaagagacatcatcaggaaaagaaagaattattgttacggaacttccttatcaggtgaacaaagcgagattaattgaaaaaatcgcagatcttgtccgagacaaaaaaatcgaaggaattaccgacctgcgagacgaatccgaccgtaacggaatgagaatcgtcattgagatccgccgtgacgccaatgctcacgtcattttgaataacctgtacaaacaaacggccctgcagacgtctttcggaatcaacctgctggcgctcgtgacgga

SEQ ID NO: 11 - DNA, 959 bp, Bacillus velezensis
  source 1..959: mol_type = genomic DNA
cctttctggactatgcaatgagcgttatcgtatcccgggcgcttccggatgtgcgtgacggtctgaagccggttcacaggcggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagatacgattgattatcaagataactatgacggcgcagaaagagaacctgtcgtcatgccttcgagatttccgaatctgctcgtaaacggagctgccggtattgcggtcggaatggcgacaaatattcctccgcatcagcttggggaagtcattgaaggcgtgcttgccgtaagtgagaatcctgagattacaaaccaggagctgatggaatacattccgggcccggattttccgactgctggtcagattttgggccggagcggcatccgcaaggcatatgaatccggacggggatcaatcacaatccgggctaaggctgaaatcgaagagacatcatcaggaaaagaaagaattattgttacggaacttccttatcaggtgaacaaagcgagattaattgaaaaaatcgcagatcttgtccgagacaaaaaaatcgaaggaattaccgacctgcgagacgaatccgaccgtaacggaatgagaatcgtcattgagatccgccgtgacgccaatgctcacgtcattttgaataacctgtacaaacaaacggccctgcagacgtctttcggaatcaacctgctggcgctcgttgacgga